(19) United States
(12) Patent Application Publication
(10) Pub. No.: US 2016/0022225 A1
(43) Pub. Date: Jan. 28, 2016

(54) DIAGNOSTIC TEST DEVICE WITH IMPROVED DISPLAY

(71) Applicant: CHURCH & DWIGHT CO., INC., Princeton, NJ (US)

(72) Inventors: Mathew Palmer, Cottenham (GB); Giles H.W. Sanders, Fowlmere (GB); Albert R. Nazareth, Mercerville, NJ (US); Timothy Snowden, Howell, NJ (US); Ovieiu Romanoschi, Highland Park, NJ (US)

(21) Appl. No.: 14/776,557

(22) PCT Filed: Mar. 5, 2014
(86) PCT No.: PCT/US2014/020512

§ 371 Date: Sep. 14, 2015

Related U.S. Application Data
(60) Provisional application No. 61/782,981, filed on Mar. 14, 2013.

(51) Int. Cl. A61B 5/00; A61B 5/15; A61B 10/00; A61B 5/145

(52) U.S. Cl. CPC A61B 5/743; A61B 5/4343; A61B 5/7405; A61B 5/7445; A61B 5/14507; A61B 5/150358; A61B 10/0051; A61B 10/007; A61B 2562/0295

(57) ABSTRACT

The present disclosure relates to diagnostic test devices that provide enhanced communication to a user thereof through provision of improved digital display. The test device can include a test member, such as lateral flow assay test strip. The test device can further include an electronic communication circuit that can comprise a digital display element as well as a microcontroller. Other elements in the electronic communication circuit can include one or more sensor elements, an audio element, and one or more switching elements. The disclosure further relates to methods of providing indicia of operation of a test device that comprises steps for assembly of a diagnostic test device that includes an improved digital display.

### CROSS-REFERENCE TO RELATED APPLICATION

This application is related to U.S. Provisional Patent Application Ser. No. 61/782,981 filed Mar. 14, 2013 and takes priority therefrom.

### FIELD OF THE DISCLOSURE

The present disclosure relates to diagnostic test devices that provide user connectivity. More particularly, the test devices include elements that provide for specific feedback to the user, such as through visual means.

### BACKGROUND

Many types of ligand-receptor assays have been used to detect the presence of various substances in body fluids, such as urine, saliva, or blood. Some commercially available assays are designed to make a quantitative determination, but in many circumstances all that is required is a qualitative positive/negative indication. Examples of such qualitative assays include blood typing, pregnancy testing, and many types of urinalysis.

U.S. Pat. No. 6,485,982, which is incorporated herein by reference in its entirety, describes a diagnostic test cell or device formed of an elongate outer casing which houses an interior permeable material (such as glass fiber) capable of transporting an aqueous solution by capillary action, wicking, or simple wetting. The casing defines a sample inlet, and interior regions, which are designated as a test volume and a reservoir volume. The reservoir volume is disposed in a section of the test cell spaced apart from the inlet and is filled with sorbent material. The reservoir acts to receive a fluid sample transported along a flow path defined by the permeable material and extending from the inlet and through the test volume. In the test volume is a test site comprising a first protein having a binding site specific to a first epitope of the ligand immobilized in fluid communication with the flow path (e.g., bound to the permeable material or to latex particles entrapped in or bonded to the permeable material). A window, such as a hole or transparent section of the casing, permits observations of the test site through the casing wall. The use of the test cell requires a conjugate comprising a second protein bound to colored particles, such as a metal sol or colloid, preferably gold. The conjugate can take two distinct forms, depending on whether the assay is designed to exploit the “sandwich” or “competitive” technique.

U.S. Pat. No. 7,045,342, which is incorporated herein by reference in its entirety, describes a diagnostic test device including a biphasic chromatographic medium. The biphasic substrate is formed of a release medium joined to a capture medium located downstream of the release medium. The release and capture media preferably comprise two different materials, or phases, having different specific characteristics. The two phases are joined together to form a single fluid path such that a solvent front can travel unimpeded from the proximal (upstream) end of the release medium to the distal (downstream) end of the capture medium.

For tests such as those described above, visually observable indicia can be preferred. Such indicia typically have included the presence of agglutination or a color change at a defined site on the assay. More recent efforts have included providing electronic (i.e., digital) signals as the observable indicia. Nevertheless, user interface with diagnostic test devices remain limited. For example, user interface disconnect can lead to user anxiety, such as in relation to uncertainty over the elapsed time between starting a test and obtaining the test result. In typical pregnancy test devices, for example, the elapsed time is typically less than five minutes. During this time, there also can be anxiety over whether the test is indeed progressing normally. For example, although some test devices include a liquid crystal display (LCD) digital readout that can display a static or blinking clock as indicia of a progressing test, such interface can be sufficiently limited so as to not meet user expectations and overcome anxiety. Another user interface disconnect can relate to user uncertainty over whether a sufficient volume of fluid sample (e.g., urine) has been applied in order for the test to progress normally and provide a valid test result. Because of these and other reasons, it would be beneficial to provide a personal use test device with improved communication between the test device and the user.

### SUMMARY OF THE DISCLOSURE

The present disclosure relates to diagnostic test devices that include elements useful for carrying out an assay and for providing information related to the assay in an informative display. As an illustrative example, a pregnancy test device can be provided and can include elements for carrying out a test on a fluid sample applied to a receiving member so as to identify the presence of human chorionic gonadotropin (hCG) in the sample that is indicative of a pregnancy status. Such test device beneficially can include further elements that enable the test device to provide a variety of visual indices relating to the operation and results of the assay. Test devices according to the present disclosure thus can provide for increased communication from the test device to a user thereof and make the test device easier for the user to operate, improve understanding of the results of the included assay, and increase user comfort with the test device and user assurance in the reliability of the test device.

In certain embodiments, a diagnostic test device according to the present disclosure can comprise a test member (for example, a test strip, particularly a strip adapted for carrying out a lateral flow assay) and an electronic communication circuit adapted to provide one or more indicia of operation of the test member to a user. In particular embodiments, the electronic communication circuit can include a color digital display.

The nature of a color digital display according to the disclosure can vary. As non-limiting examples, the display can be defined as follows: the color digital display can comprise a plurality of color-variable pixels; the color digital display can comprise one or more light emitting diode (LED), particularly an organic light emitting diode (OLED); the color digital display can comprise a thin film transistor; and the color digital display can comprise a liquid crystal display (LCD).

In some embodiments, an LCD can be defined by certain characteristics. For example, the LCD can define a plurality of switchable liquid crystal characters positioned in an opaque field, and the characters can be adapted to provide the one or more indicia of operation of the test member. The switchable liquid crystal characters can be adapted to independently make visible one or more of the characters by transitioning between a transparent state that provides a visible light path corresponding to each of the respective characters and an opaque state that is substantially visually identical to the opaque field. In addition, the LCD can comprise a color filter positioned in the visible light path. The LCD can comprise a light reflective member positioned behind the plurality of switchable liquid crystal characters. The LCD optionally can comprise backlighting.

The electronic communication circuit of the diagnostic test device preferably can comprise a microcontroller, and the microcontroller can be adapted to signal the color digital display to make visible one or more colored characters corresponding to the one or more indicia of operation of the test member. Beneficially, the electronic communication circuit further can comprise a memory component, and such memory component can comprise code defining the one or more colored characters in reference to the indicia of operation of the test member. The memory component can be part of the microcontroller, and/or the memory component can be separate from the microcontroller (e.g., an embodiment wherein the microcontroller includes a memory component and where a second memory component is otherwise included in the electronic communication circuit).

The characters displayed by the color digital display of the diagnostic test device can vary. Moreover, the number of characters that can be displayed on the color digital display can vary. As an example, the characters can be selected from the group consisting of letters, numbers, symbols, pictures, and combinations thereof. In some embodiments, the microcontroller can be adapted to signal the color digital display to indicate one or more times information related to the diagnostic test device (e.g., by displaying the appropriate characters).

In some embodiments, the color digital display can be a static display wherein all characters are defined in a specific location on the display screen, said location being unchangeable. In other embodiments, the color digital display can be a dynamic display wherein one or more characters can be displayed so as to simulate motion of the character on the display screen.

In the diagnostic test device of the disclosure, the electronic communication circuit further can comprise one or more components adapted to provide input signals relating to the test to the microcontroller. The microcontroller can parse the signal and direct an output defining a character or characters to be shown by the color digital display, if any. The signal can arise from a variety of components of the diagnostic test device (e.g., optodetectors, moisture sensors, and the like). Further, the test device can comprise one or more components adapted to carry out a test initiation routine. The electronic communication circuit further can include an audio output component. The electronic communication circuit also can comprise a power source.

The diagnostic test device of the disclosure can be adapted to detect the presence of an analyte in a fluid sample applied to the test member. For example, an analyte to be detected can be selected from the group consisting of human chorionic gonadotropin (hCG), luteinizing hormone (LH), follicle stimulating hormone (FSH), thyroid stimulating hormone (TSH), estrogen, progesterone, testosterone, a metabolite thereof, and combinations thereof. Other analytes—e.g., proteins—can also be detected by the presently disclosed test device. To facilitate such detection, the test member used in the diagnostic test device can comprise a release medium in fluid communication with a capture medium. Examples include a biphasic test strip and a triphasic test strip.

In further embodiments, a diagnostic test device according to the disclosure can comprise components adapted to provide further functionality to the device. In some embodiments, a diagnostic test device can comprise a test member and an electronic communication circuit adapted to provide one or more indicia of operation of the test member to a user. In particular, the electronic communication circuit can include a dynamic digital display, specifically a dynamic digital display that is adapted to render variable text characters.

In certain embodiments, the dynamic digital display can be adapted to display one or more characters in a sequence that simulates motion on the display. As non-limiting examples, the one or more characters can be selected from the group consisting of letters, numbers, symbols, pictures, and combinations thereof. In particular embodiments, the one or more characters can define a word or a string of words—e.g., a string of words that impart information to a user in reference to the diagnostic test device. Preferably, the sequence in which the characters are displayed can define scrolling of the characters on the display. Further, the electronic communication circuit of the diagnostic test device can comprise a microcontroller.

The components defining the simulated motion on the display can vary. For example, the dynamic digital display can comprise a plurality of multi-pixelated fields which preferably can be in a side-by-side arrangement (which encompasses horizontal and vertical arrangements). Each multi-pixelated field can be adapted to display a single character at a time.

The electronic communication circuit further can comprise a memory component. The memory component can comprise code that defines sequences of characters that define words that can be displayed in pre-determined sequences that impart information related to the diagnostic test device. The microcontroller can be adapted to signal the multi-pixelated fields to move the sequences of characters in a single direction to adjacent multi-pixelated fields at a defined time interval so as to simulate scrolling or other motion of the words on the dynamic digital display. The memory component can be part of the microcontroller, and/or the memory component can be separate from the microcontroller (e.g., an embodiment wherein the microcontroller includes a memory component and where a second memory component is otherwise included in the electronic communication circuit). A memory component particularly can be part of a driver used in connection with the dynamic digital display.

In the diagnostic test device of the present disclosure, the electronic communication circuit further can comprise one or more components adapted to provide input signals relating to the test to the microcontroller. The microcontroller can parse the signal and direct an output defining a character or characters to be shown by the digital display, if any. The signal can arise from a variety of components of the diagnostic test device (e.g., optodetectors, moisture sensors, and the like). Further, the test device can comprise one or more components adapted to carry out a test initiation routine. The electronic communication circuit further can include an audio output component. The electronic communication circuit also can comprise a power source.

A diagnostic test device comprising a dynamic digital display can be adapted to detect the presence of an analyte in a fluid sample applied to the test member. For example, an analyte to be detected can be selected from the group consisting of human chorionic gonadotropin (hCG), luteinizing hormone (LH), follicle stimulating hormone (FSH), thyroid stimulating hormone (TSH), estrogen, progesterone, testosterone, a metabolite thereof, and combinations thereof. Other analytes—e.g., proteins—can also be detected by the presently disclosed test device. To facilitate such detection, the test member used in the diagnostic test device can comprise a release medium in fluid communication with a capture medium. Examples include a biphasic test strip and a triphasic test strip.

In still further embodiments, a diagnostic test device of the present disclosure can be characterized in relation to a sequential illumination of characters on a display. In particular, a diagnostic test device according to some embodiments of the disclosure can comprise a test member and an electronic communication circuit adapted to provide one or more indicia of operation of the test member to a user. Specifically, the electronic communication circuit can comprise a digital display adapted for progressive illumination of a plurality of characters and a microcontroller adapted to signal the digital display to progressively illuminate or darken the characters in a defined sequence. The progressive illumination can proceed at pre-determined time intervals. Likewise, the defined sequence of the progressive illumination can define a period of time. Such time period can be related to the time during which one or more stages of a test is carried out by the diagnostic test device. In various embodiments, the progressive illumination can define a count down of the period of time, or the progressive illumination can define a count up of the period of time. If desired, the digital display can be adapted to display the plurality of characters in color, and the plurality of characters can be displayed in two or more different colors.

The nature of the digital display can vary. As non-limiting examples, the display can be defined as follows: the digital display can comprise one or more light emitting diode (LED), particularly an organic light emitting diode (OLED); the digital display can comprise a thin film transistor; the digital display can comprise a liquid crystal display (LCD); and the digital display can comprise a plurality of multi-pixelated fields.

In the diagnostic test device of the present disclosure adapted for progressive illumination, the electronic communication circuit further can comprise one or more components adapted to provide input signals relating to the test to the microcontroller. The microcontroller can parse the signal and direct an output defining a character or characters to be shown by the digital display, if any. The signal can arise from a variety of components of the diagnostic test device (e.g., optodetectors, moisture sensors, and the like). Further, the test device can comprise one or more components adapted to carry out a test initiation routine. The electronic communication circuit can include an audio output component. The electronic communication circuit also can comprise a power source.

A diagnostic test device comprising progressive illumination can be adapted to detect the presence of an analyte in a fluid sample applied to the test member. For example, an analyte to be detected can be selected from the group consisting of human chorionic gonadotropin (hCG), luteinizing hormone (LH), follicle stimulating hormone (FSH), thyroid stimulating hormone (TSH), estrogen, progesterone, testosterone, a metabolite thereof, and combinations thereof. Other analytes—e.g., proteins—can also be detected by the presently disclosed test device. To facilitate such detection, the test member used in the diagnostic test device can comprise a release medium in fluid communication with a capture medium. Examples include a biphasic test strip and a triphasic test strip.

The present disclosure also encompasses methods for providing one or more indicia of operation of a test to a user. Such methods can include one or more steps defining the formation of a diagnostic test device as discussed herein.

In some embodiments, a method for providing one or more indicia of operation of a test device to a user can comprise combining, in a single casing: a test member; and an electronic communication circuit that includes: a microcontroller; one or more components adapted to provide input signals to the microcontroller relating to the test member; and a color digital display. The method further can comprise programming the microcontroller to respond to a defined input signal by signaling the color digital display to make visible one or more color characters defining the one or more indicia of operation of the test. Specifically, the character can be selected from the group consisting of letters, numbers, symbols, pictures, and combinations thereof.

In other embodiments, a method for providing one or more indicia of operation of a test device to a user can comprise combining, in a single casing: a test member; and an electronic communication circuit that includes: a microcontroller; one or more components adapted to provide input signals to the microcontroller relating to the test member; and a dynamic digital display. In particular, the dynamic digital display can comprise a plurality of multi-pixelated fields. The method also can comprise programming the microcontroller with code that defines sequences of characters that define words. The method further can comprise programming the microcontroller to respond to a defined input signal by signaling the dynamic digital display to display the characters in pre-determined sequences that impart information defining the one or more indicia of operation of the test.

In further embodiments, a method for providing one or more indicia of operation of a test device to a user can comprise combining, in a single casing: a test member; and an electronic communication circuit that includes: a digital display adapted for progressive illumination of a plurality of characters; a microcontroller adapted to signal the digital display to progressively illuminate or darken the characters in a defined sequence; and one or more components adapted to provide input signals to the microcontroller relating to the test member. In particular embodiments, the progressive illumination can proceed at pre-determined time intervals. Further, the defined sequence of the progressive illumination can define a period of time. The method can be characterized in that the input signal can define the start of a test or a stage thereof. The method further can comprise programming the microcontroller to respond to the input signal by signaling the digital display to progressively illuminate or darken the characters over a time period defined by the amount of time for completion of the test or the stage thereof.

### BRIEF DESCRIPTION OF THE DRAWINGS

The present disclosure is particularly described in reference to the following figures; however, such figures are provided to illustrate only preferred embodiments of the disclosure, and the disclosure is not intended to be limited thereto.

FIG. 1A shows a perspective view of an analog detection device according to one exemplary embodiment of the disclosure;

FIG. 1B shows a perspective view of the detection device of FIG. 1A with a disengaged cap;

FIG. 1C shows a top view of the detection device of FIG. 1A;

FIG. 2A shows a perspective view of a digital detection device according to one exemplary embodiment of the disclosure;

FIG. 2B shows a perspective view of the detection device of FIG. 2A with a disengaged cap;

FIG. 2C shows a top view of the detection device of FIG. 2A;

FIG. 3A shows a top view of lateral flow test components according to one exemplary embodiment of the disclosure comprising a reservoir absorbent material, a biphasic substrate, and a sample receiving member outside of a casing;

FIG. 3B shows a top view of a biphasic substrate for use in a test device according to an exemplary embodiment of the disclosure;

FIG. 4A shows a top view of a lateral flow test strip comprising a triphasic substrate according to an exemplary embodiment of the disclosure;

FIG. 4B shows a side view of the triphasic substrate of FIG. 4A;

FIG. 5 is a schematic of operational components of an electronic communication circuit of a test device according to one exemplary embodiment of the disclosure, the electronic communication circuit being arranged to provide desired display characteristics;

FIG. 6 is an illustration of a digital display screen adapted to provide location specific display of a defined set of characters;

FIG. 7 is an illustration of a digital display screen according to an exemplary embodiment of the disclosure defining a plurality of multi-pixelated fields;

FIG. 8a is an illustration of a digital display screen according to an exemplary embodiment of the disclosure defining a plurality of bar segments that are illuminated or darkened and that combine to form a progress bar;

FIG. 8b is an illustration of a digital display screen according to an exemplary embodiment of the disclosure defining a plurality of wheel sections that are illuminated or darkened and that combine to form a progress wheel;

FIG. 9 is a schematic of operational components of an electronic communication circuit of a test device according to an exemplary embodiment of the disclosure, the electronic communication circuit being arranged to provide desired display characteristics;

FIG. 10 is a flow chart illustrating an exemplary embodiment of a method for providing one or more indicia of operation of a lateral flow assay to a user through formation of an appropriate test device comprising a color digital display;

FIG. 11 is a flow chart illustrating an exemplary embodiment of a method for providing one or more indicia of operation of a lateral flow assay to a user through formation of an appropriate test device comprising a dynamic digital display; and

FIG. 12 is a flow chart illustrating an exemplary embodiment of a method for providing one or more indicia of operation of a lateral flow assay to a user through formation of an appropriate test device comprising a progressive illumination digital display.

### DETAILED DESCRIPTION OF THE DISCLOSURE

The present disclosure now will be described more fully hereinafter with reference to specific embodiments and particularly to the various drawings provided herewith. Indeed, the disclosure may be embodied in many different forms and should not be construed as limited to the embodiments set forth herein; rather, these embodiments are provided so that this disclosure will satisfy applicable legal requirements. As used in the specification, and in the appended claims, the singular forms “a,” “an,” “the,” include plural referents unless the context clearly dictates otherwise.

In one aspect, the present disclosure relates to a test device, such as an over-the-counter (OTC) or point of care (POC) test device, for detecting an analyte in a sample. The device generally includes components suitable for carrying out an assay, such as a lateral flow assay, and also includes components suitable for communicating information relating to the assay to an individual.

The test components in a broad sense can comprise a proximal portion (e.g., a sample receiving member) in fluid communication with a distal portion (e.g., a reservoir). The proximal and distal portions may be interconnected by a substrate material, which itself may form all or part of the proximal and/or distal portion of the device. A sample (e.g., urine) can be directly or indirectly applied to the proximal portion of the device for transport to the distal portion. Preferably, the sample flows across the substrate so as to contact one or more antibodies attached to or otherwise deposited on the substrate. The antibodies can be designed and/or chosen to recognize a variety of analytes. In specific embodiments, a test device according to the present disclosure can be useful for detection of human chorionic gonadotropin (hCG), luteinizing hormone (LH), follicle stimulating hormone (FSH), thyroid stimulating hormone, estrogen, progesterone, testosterone, a metabolite thereof, and combinations thereof. Even further analytes also can be encompassed by the present disclosure.

The devices disclosed herein can make use of a variety of techniques for detecting the presence of an analyte. One example is a sandwich technique wherein one or more antibodies used in the detection comprise a binding member or site which binds to an epitope on the analyte for detection. A labeled antibody binds to the analyte to form a complex in the sample. The analyte, which is bound to the labeled antibody or antibodies, binds with one or more capture antibodies to form a “sandwich,” comprising the capture antibody, analyte (or antigen), and the labeled antibody. Each sandwich complex thus produced comprises three components: one capture antibody, one antigen, and one labeled antibody. An antibody used herein can be a polypeptide substantially encoded by an immunoglobulin gene or immunoglobulin genes, or fragments thereof, which may specifically recognize and bind an antigen. The recognized immunoglobulin genes include the kappa, lambda, alpha, gamma, delta, epsilon, and mu constant region genes, as well as the immunoglobulin variable region genes. Antibodies include fragments, such as Fab′, F(ab)₂, Fabc, and Fv fragments. The term antibody also can include antibody fragments either produced by the modification of whole antibodies or those synthesized de novo using recombinant DNA methodologies, and further can include “humanized” antibodies made by conventional techniques. Although polyclonal antibodies can be used, antibodies are preferably monoclonal antibodies. A capture antibody according to the disclosure can be an antibody attached to a substrate directly or indirectly, such as a solid substrate. The capture antibody specifically or preferentially binds a particular distinct epitope of an antigen.

In the sandwich technique, the makeup of each sandwich complex can vary depending upon the particular labeled antibody (and thus the particular antigen) included therein. In the same test, there can be multiple different types of sandwiches produced. The sandwich complexes are progressively produced as the test sample with the analyte therein continuously moves along the substrate of the device. As more and more of the analyte/labeled antibody complex is immobilized in sandwich form with the capture antibody or antibodies at the capture site, the label components aggregate and become detectable in that the accumulation of the sandwich complexes at the capture site can be detected in various ways, such as by visual inspection of, for example, color development at the capture site or by a digital readout resulting from the electronic analysis of the aggregate at the capture site as further described herein. Although the sandwich technique is provided as an exemplary embodiment, the devices described herein in relation to the improved communication aspects are not limited to such underlying technique. Rather, other techniques for identifying an analyte in a test sample and forming a detectable signal based on the presence or absence of the analyte in the sample can be utilized.

Exemplary means for forming a detectable signal can comprise the use of a conjugate comprising one or more antibodies bound to detectable label components (e.g., colored particles, such as a metal sol or colloid particles). One or more of the antibodies used in the disclosed devices (e.g., one or two) can be labeled. Any detectable label recognized in the art as being useful in various assays can be used. In particular, the detectable label component can include compositions detectable by reflective, spectroscopic, photochemical, biochemical, immunochemical, or chemical means. As such, the label component produces a detectable signal. For instance, suitable labels include soluble dyes, fluorescent dyes, chemiluminescent compounds, radioisotopes, electron-dense reagents, enzymes, colored particles, or dioxigenin. The label component can generate a measurable signal, such as radioactivity, fluorescent light, color, or enzyme activity, which can be used to identify and quantify the amount of label bound to a capture site. Thus, the label component can also represent the presence or absence of a particular antigen bound thereto, as well as a relative amount of the antigen (e.g., relative to a known standard, threshold standard, or a different standard). The labeled materials can be detected through use of suitable electronic components, including hardware and software, and thus can be communicated to a user via digital signal or similar means. Further detail regarding the production of digital signals in personal use assays is provided, for example, in U.S. Pat. No. 7,214,542 to Hutchinson; U.S. Pat. No. 7,220,597 to Zin et al.; and U.S. Pat. No. 7,499,170 to Sasaki et al., which are incorporated herein by reference.

Devices according to the present disclosure can include one or more standards or internal controls that allow for determination of whether signal development is a true indication of the presence or absence of analyte in the sample or is simply an artifact, such as caused by nonspecific sorption. For example, a negative control site can be prepared identically to the test site, except that immobilization of the capture antibody is omitted. Therefore, although the conjugate will reach the negative control site, it will aggregate due only to non-specific binding. Similarly, the device can include a positive control, such as with an authentic sample of the analyte for detection immobilized at the positive control site. An alternate control site can be located downstream of the capture site and have immobilized thereon at least one capture protein (e.g., an antibody). Such control site can function to capture and immobilize labeled antibody which has not been captured at the capture site. For example, such control site can include polyclonal antisera specific for the labeled antibody immobilized thereon to indicate proper functioning of the assay.

In some embodiments, a biphasic chromatographic medium (substrate/test strip) can be used in the disclosed assays and can comprise an upstream release medium joined to a downstream capture medium. The release and capture media can comprise two different materials or phases having different specific characteristics. The two phases can be joined together to form a single fluid path such that a solvent front can travel unimpeded from the proximal (upstream) end of the release medium (which can be defined as a proximal portion of the biphasic medium) to the distal (downstream) end of the capture medium (which can be defined as a distal portion of the biphasic medium). A sample receiving member can be generally provided at the proximal end of the biphasic substrate and a reservoir of sorbent material can be located beyond the biphasic substrate.

In other embodiments, a triphasic chromatographic medium (substrate/test strip) can be used in the disclosed assays and can comprise a capture medium overlapped at one end by a release medium and at the opposing end by a reservoir. The triphasic substrate can be in fluid communication with a sample receiving member at the end thereof comprising the release medium.

In certain embodiments, use of a biphasic or triphasic chromatographic medium may enhance the speed and sensitivity of an assay, such as those described in U.S. Pat. No. 6,319,676, U.S. Pat. No. 6,767,714, U.S. Pat. No. 7,045,342, and U.S. Publication No. 2012/0083044, which are incorporated herein by reference, including without limitation for the purpose of describing biphasic and triphasic chromatographic media. Methods for manufacturing chromatographic media are also described in detail in U.S. Pat. No. 5,846,835, the disclosure of which is incorporated herein by reference in its entirety.

Reagents for detecting, labeling, and capturing an analyte of interest can be disposed on the release and capture media. In certain embodiments, one or more labeled conjugates can be located on the release medium and each can include a binding member (e.g., antibody) that may be reactive with a particular site (sometimes referred to as a “first epitope,” “second epitope,” etc.) on the analyte of interest. The labeled conjugates further can comprise one or more detectable markers (or labels), as discussed herein.

The release medium can be formed from a substance which allows for release of reagents deposited thereon, which can comprise reagents that are releasably (i.e., not permanently) bound to the release medium. The primary function of the release medium is first to support and to subsequently release and transport various immunological components of the assay, such as a labeled conjugate and/or a capturable conjugate, both of which are capable of binding to the analyte of interest. The release medium can be formed of any material capable holding, releasing, and transporting various immunological parts of the test such as the labeled test component (e.g., a bibulous, hydrophilic material).

The capture medium can be formed from a material which permits immobilization of reagents for detection of the presence of analyte in the test fluid. Immobilization can refer to any interaction that results in antibodies or analytes being irreversibly bound to the substrate such that they are not appreciably washed away, e.g., during the course of a single use of the device. The capture medium can comprise hydrophilic polymeric materials, such as microporous films or membranes, which permit protein reagents to be immobilized directly on the membrane by passive adsorption without the need for chemical or physical fixation, although fixation such is not excluded.

The release medium and capture medium can be joined via any suitable means. For example, the two media can be joined by overlapping the downstream edge of the release medium over the upstream edge of the capture medium. The various media components of the biphasic or triphasic substrate can be adhered to a clear polymer film or opaque sheet, thereby holding the media in place. Alternately, the media can be connected by a non-overlapping butt joint and may still be attached to an underlying support.

The diffusible and non-diffusible reagents can be applied to the release and capture media, respectively, by any suitable technique. In one embodiment, the diffusible antibody reagents can be applied to the release medium by direct application onto the surface of the medium and dried to form a band. Generally, reagents can be immobilized using absorption, adsorption, or ionic or covalent coupling, in accordance with any suitable methods.

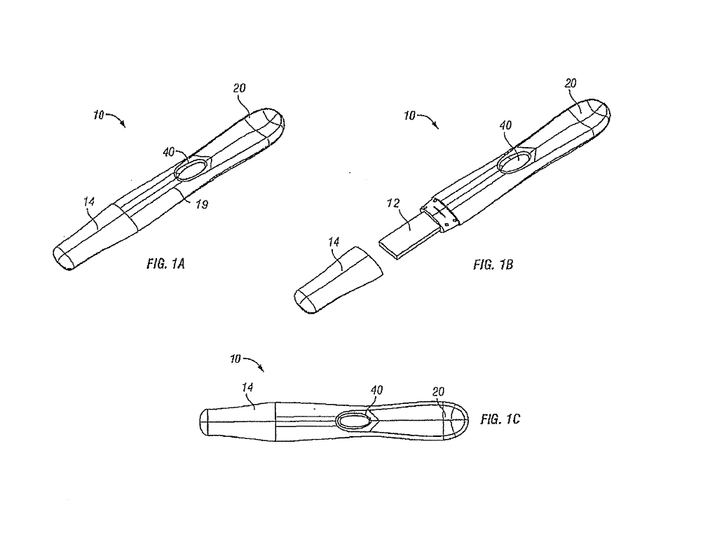

In one embodiment, a test device 10 according to the present disclosure can comprise a casing defining a sample inlet, a test volume, and reservoir volume, as illustrated in FIG. 1A-FIG. 1C. The casing 19 is generally configured to provide a recessed portion 20 shaped to permit users to place their thumb into the recessed portion and their forefinger on the bottom of the casing to securely hold the test device 10. Disposed within the casing 19 are the functional components forming a test member. The test member can be a single strip or a combination of strips of materials useful for providing an assay. For example, the test member can be a test strip as described herein, such as comprising a biphasic or triphasic substrate, for use in an assay. A sample receiving member 12 can be disposed within the casing 19, extends to the exterior thereof, and may be covered by a removable cap 14.

In use, a test sample passes from the sample receiving member 12 to a test member, such as a chromatographic substrate, where the sample is in reactive contact with the test site (e.g., the capture site), and optionally one or more control sites. A central display section 40 on the top of the casing defines a region that permits a user to observe test results as they become detectable. As described herein, “becoming detectable” specifically can relate to the accumulation of sandwich complexes at the capture site, which can be detected in various ways, such as by visual inspection of, for example, a digital readout resulting from the electronic analysis of the aggregate at the capture site as further described herein. In the embodiment illustrated, the display section 40 can provide for viewing of an analog signal, such as a colored indicator of accumulation of labeled complexes at the test site visible through the display section. Although not expressly shown, the test device 10 further can include one or more perforations in the casing to improve audibility of any audio communications that are provided from the test device, as otherwise described herein. Such perforations can be present, for example, in the location of a loudspeaker or other audio component of the test device. Further, components necessary to form an electronic communication circuit can be retained with the casing of the test device, as otherwise described herein. More particularly, one or a series of holes, slots, or the like can be present to improve sound transmission from the device.

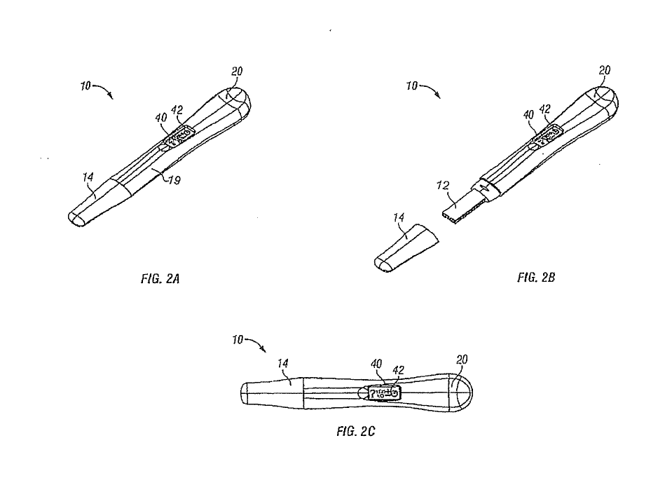

A further embodiment is illustrated in FIG. 2A-FIG. 2C. Again, the test device 10 is formed of a casing 19 that comprises a recessed portion 20 at a back portion thereof and that comprises a sample receiving member 12 at a front portion thereof that extends into the casing. The sample receiving member 12 preferably is covered by a removable cap 14. In this embodiment, the central display section 40 includes an underlying display screen 42 that can provide digital displays of one or more statuses and results of the test device. The display screen can be structured and adapted for providing visible signals as further described herein. The display screen (and thus the display section) can be sized or shaped as illustrated or may vary as useful for display of desired information. The casing 19 can enclose a suitable test member (e.g., a biphasic or triphasic substrate and associated elements, such as a reservoir).

In the use of one exemplary assay, a sample passes through the inlet defined by the sample receiving member and into the interior of a device, where it comes into contact with the test member including a release medium and a capture medium. If the analyte of interest is present in the sample, it binds to the one or more labeled antibodies which are releasably attached to the release medium. The sample, now comprising analyte-labeled antibody conjugates, wicks up the release medium and forms a sandwich complex with one or more capture antibodies immobilized on the capture medium (defining a capture site or test site). As the sample front passes across the capture site, a complex is formed comprising the analyte, labeled antibody, and the capture antibody. This “sandwich” complex can be analyzed by detecting the presence of the label at the capture site. Detection can be via a visual review of a formed color in the display section of an analog device or via viewing a digital readout on, for example, an LCD screen of a digital device. As further discussed herein, detection also can include audible signals or words. Although the present disclosure is described largely in terms of direct devices/direct detection, other devices (i.e., affinity-based devices) are also intended to be encompassed herein. Affinity-based devices operate on similar principles, but rely on indirect binding (wherein one member of an affinity pair (e.g., biotin) is present on a capturable conjugate (and subsequently on any diffusible sandwich complex formed therefrom) and the other member of the affinity pair (e.g., avidin) is present on the capture medium section of the substrate).

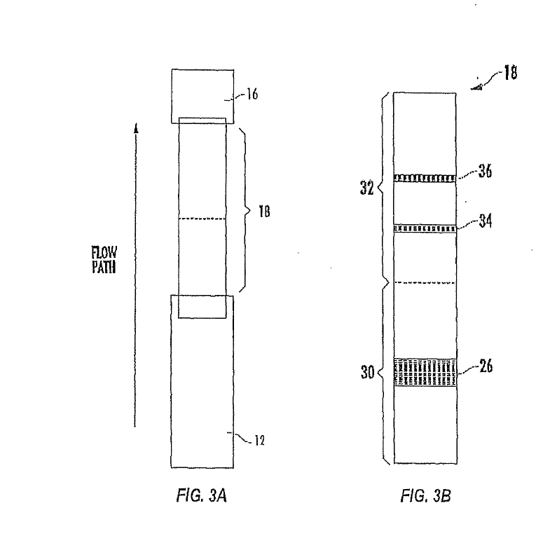

FIG. 3A shows an example of lateral flow test components that can be present in a device such as illustrated in FIG. 1A-FIG. 1C. These test components can comprise a sample receiving member 12, biphasic chromatographic substrate 18, and reservoir absorbent material 16. When the device is placed in contact with a fluid sample, the fluid is transported by capillary action, wicking, or simple wetting along the flow path downstream through sample receiving member 12, along chromatographic substrate 18, and into reservoir absorbent material 16, generally as depicted by the arrow. Sample receiving member 12 may also serve as a filter which can remove particulate matter and interfering factors from a sample. The sample receiving member 12 preferably is a bibulous hydrophilic material which facilitates absorption and transport of a fluid sample to the biphasic chromatographic substrate 18. Such materials may include cellulose acetate, hydrophilic polyester, or other materials having similar properties. A combination of absorbent materials also may be used. As noted above, a filtration means which limits the introduction to the test site of contaminants from the sample may also be included. In certain embodiments, the sample receiving member 12 can be omitted, and the release medium of a biphasic substrate 18 can itself act as the sample receiving member. Such embodiments of the assay materials are useful in performing dipstick assays. By providing a reservoir of sorbent material (e.g., absorbent paper made from cotton long linter fibers or cellulosic materials) disposed beyond the chromatographic substrate, a relatively large volume of the test fluid and any analyte it contains can be drawn through the test area to facilitate background clearance and thereby aid sensitivity. The reservoir absorbent generally facilitates capillary action along the chromatographic substrate and absorbs excess fluid contained within the device.

FIG. 3B illustrates in greater detail an exemplary biphasic chromatographic substrate 18, comprising a release medium 30 and a capture medium 32 joined together to form a single fluid path. A band 26 of labeled binding member, e.g., an antibody-metal sol, can be releasably disposed on the release medium 30. In one embodiment, the labeled binding member is in dehydrated form. As the fluid sample moves past the band 26, the labeled binding member becomes entrained in the fluid, reconstituted (in the case of a dehydrated binding member), and binds with a particular analyte or analytes of interest present in the fluid sample. Accordingly, the resulting complex comprising a binding antibody, a label component, and an analyte for identification (e.g., hCG) advances along with the sample front until it reaches the capture site 34. In this particular embodiment, the capture site includes at least one immobilized capture antibody which binds to a different epitope of the analyte. Accordingly, a sandwich complex including the desired analyte is formed at the capture site 34. If desired, a control site 36 can be present.

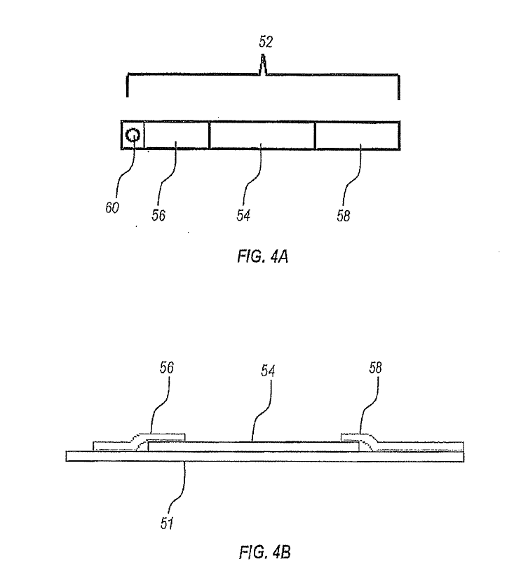

A further exemplary lateral flow test strip that can be present in a device according to the present disclosure is illustrated in FIG. 4A. In particular, a triphasic test strip 52 is shown and is formed of a release medium 58, a capture medium 54, and a reservoir 56. An alignment hole 60 is shown and can be used to align the test strip within a casing by mating with an appropriately positioned pin. FIG. 4B illustrates an overlapping relation of the release medium 58, capture medium 54, and reservoir 56. Although not illustrated, the release medium 58 can be in fluid communication with a sample receiving member as already described herein. Further, the release medium 58, capture medium 54, and reservoir 56 can be laminated onto a backing 51, which can be, for example, an opaque plastic film or sheet. In use, the appropriate antibodies, binding members, and labels can be positioned on the release medium 58 and the capture medium 54, and an advancing fluid sample can cause formation of a complex, such as, for example, the combination of a binding antibody, a label component, and an analyte for identification. This complex then can bind with a binding member on the capture medium 54. The resulting, bound complex can be analyzed by the detection means as otherwise discussed herein, and a result then can be provided via a digital display, for example, an LCD, visible through the central display section 40. The release and capture media can be constructed of materials as described above in relation to a biphasic substrate embodiment.

For further detail regarding various testing devices, methods of use, and parameters thereof, see for example U.S. Pat. Nos. 5,739,041; 6,046,057; 6,277,650; 6,319,676; 6,767,714; 7,045,342, 7,763,454; 7,776,618 and 8,211,711 to Nazareth et al., and U.S. Patent Application Publication Nos. 2002/0042082, 2004/0171174; 2008/0213920; 2010/0051350; 2010/0239460; 2010/0240149; 2010/0261293; 2010/0267166; and 2011/0201122 to Nazareth et al., and 2012/0083044 to Sturman et al.; which are incorporated herein by reference in their entireties.

Communication components of the test devices disclosed herein beneficially can provide for improvements not only in communicating test results but also in providing additional information to a user that can impart an improved sense of connectivity of the user with the device. A variety of indices of operation can be communicated to a user during and/or after use of the present devices. In certain embodiments an indication of the functioning of the device and/or the results of the test being carried out can be identified to the user by providing one or more digital outputs, including but not limited to words, symbols, sounds, and colors that may be read or otherwise interpreted by the user. Such embodiments can comprise, for example, an opto-electronic reader coupled with a software program which can evaluate various parameters (e.g., chemical parameters or physical parameters) at the capture site. The program software can provide additional functions, as otherwise described herein. A variety of digital outputs thus can be encompassed by the devices disclosed herein, and such outputs can communicate multiple different types of information to a user of the device.

In specific embodiments, a test device according to the present disclosure can comprise assay components, such as those described herein, that are effective to carry out a diagnostic test on a fluid sample and identify (qualitatively as well as quantitatively) the presence of an analyte in the sample. The test device also can comprise at least one electronic control component (e.g., a microcontroller) and at least one digital display. Further, the test device can comprise one or more components adapted to provide input signals to the microcontroller relating to the assay. Moreover, the microcontroller can include programming such that the microcontroller is adapted to respond to a defined input signal by signaling the digital display to make visible a corresponding signal. These elements of a test device according to various embodiments of the present disclosure are discussed in greater detail below.

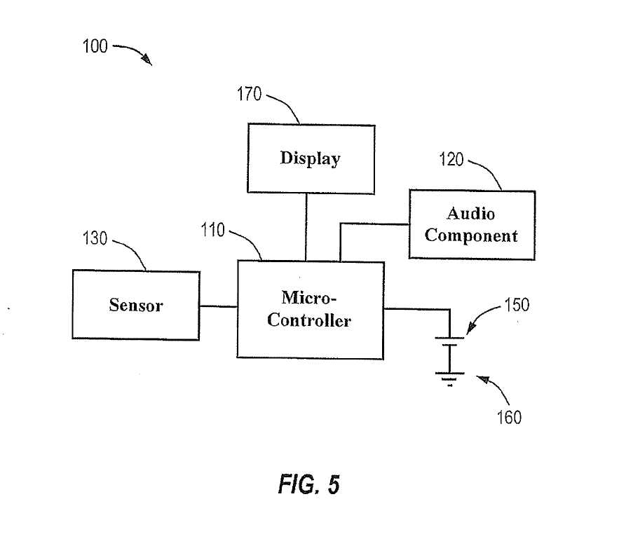

In relation to certain embodiments, a schematic of operational components of an electronic communication circuit 100 useful according to the present disclosure is shown in FIG. 5. The communication circuit utilized in the test device generally can include all components necessary to generate an input signal, process the input signal, direct a defined output signal in response to the input signal, and execute the output signal so as to deliver a visual indicia of an operation of the test device. The communication circuit can include a microcontroller 110 that is adapted to direct the overall functions of the test device, process information about the assay (such as described above), and output signals to cause formation of visual messages that can be understood or interpreted by a user. If desired, a microprocessor may be utilized so long as any further hardware or software necessary to carry out the functions of the test device are included as well (e.g., RAM or ROM). Controllers and processors that are commercially available can be adapted for use according to the present disclosure. Preferably, the controller includes programming (e.g., embedded software) that includes the requisite definitions of input signals and associated output signals to enable to the controller to direct the communication functions of the test device, as otherwise disclosed herein.

In certain embodiments, output signals on the display can be provided in one or more colors in the visual spectrum. Specifically, a color display according to the present disclosure can encompass a display providing a signal, message, or the like in a color outside the set of black or white. Thus, the presently disclosed devices provide a display that is not limited to binary images wherein the only two possible values for a pixel or other display segment are black and white (or an approximation thereof). The present displays do not necessarily exclude the use of black and white as displayable colors but rather comprise black and white (including grayscale) in the full spectrum of colors, or any portion thereof, that may be provided.

A diagnostic test device according to the present disclosure further can include one or more components adapted to provide input signals relating to the test to the microcontroller. For example, referencing FIG. 5, the communication circuit 100 further can include a sensor 130 that is adapted to detect a condition, signal, state, or result of the associated test and thus form an input signal. The sensor or detector is electrically connected to the microcontroller 110 such that the microcontroller can process the input signal from the sensor and act accordingly thereon. A variety of sensors can be used according to the present disclosure. For example, opto-electronic detectors and similar combinations of light sources (e.g., LEDs) and light sensors can be used to electronically read a signal formed at a test site, such as through agglomeration of labeled antibodies, as discussed above. Such sensors can provide a qualitative input signal (i.e., a yes/no or I/O based upon whether a detectable signal is or is not formed at the test site) and can also provide a quantitative input signal (i.e., estimating an analyte concentration based upon a programmed reference value, such as can be estimated based upon color or color intensity at a test site). Such detectors also can be utilized to identify application of a fluid sample to a portion of the assay, such as a sample receiving member 12 (referencing FIG. 2B), a release medium 58 (referencing FIG. 4B), or a capture medium 54 (referencing FIG. 4B). In other embodiments, moisture sensors can be used to detect the application of the fluid sample, and such sensors can be calibrated to provide the input signal only when a defined moisture level is attained so as to be indicative of the application of a sufficient volume of fluid for the assay to proceed according to normal operation. The moisture sensor can be located in fluid communication with one or more of the sample receiving member, the release medium, and the capture medium in order to determine if sufficient sample has been applied to drive the assay to completion.

The communication circuit 100 further can include an optional audio component 120 that is electrically connected to the microcontroller 110 such that the microcontroller can deliver output signals to the audio component for execution. The nature of the audio component can in part be based upon the nature of the audio communication to be delivered by the test device. For example, in certain embodiments, the audio component can be configured to deliver and output sound that can be selected from the group consisting of a single tone, a series of tones, a melody, a synthesized word or words, a recorded word or words, and combinations thereof. Audio elements necessary to achieve one or more of these outputs can be included in the test device. In some embodiments, the audio component can comprise a transducer. For example, a surface-mount audio transducer can be provided on an integrated circuit (including the same IC as the microcontroller, if desired), and the selection thereof can vary based upon the desired sound pitch, response time, voltage, sound that is output, and size. Such devices and elements are further disclosed in co-pending U.S. Pat. App. No. 61/779,615, the disclosure of which is incorporated herein by reference in its entirety.

The microcontroller can determine when to generate the appropriate output signal depending upon the state of the assay. As a non-limiting example, the communication circuit 100 can include an assay initiation routine (e.g., as part of the embedded software in the microcontroller 110) that can be triggered by an on switch 160. For example, the removal of the cap 14 from the test device 10 can automatically trigger power to flow from the power source 150 (e.g., a battery) to further components of the communication circuit. Such trigger may be via mechanical or optical (i.e., a light sensor) means, and the switch 160 can vary accordingly and can be independent of the cap. This can function as an input signal to the microcontroller to generate an output signal to the display to output a visual signal that indicates that the test is activated. Similarly, when a moisture sensor indicates that a sufficient volume of fluid sample has been deposited on the test strip, the microcontroller can generate an output signal to the display to provide a visual message that the requisite fluid sample has been applied. Likewise, a variety of words, symbols, or the like can be visually displayed periodically while the assay is being carried out and/or when the assay is complete and a test result is available. For example, while the test is processing, a character or a plurality of characters can be displayed as indication of the processing status and an estimation of the amount of time left to completion of the test or a stage thereof. Such characters can include a single character (e.g., a clock face) with one or more elements thereof in motion (e.g., a clock hand moving around the clock face) or can include a plurality of character that are displayed or illuminated in a sequence.

In relation to the various display embodiments of the present disclosure, reference can be made to the test that is carried out using the diagnostic test device or a “stage” of the test. A test “stage” is understood to encompass the entirety of the test from start to completion (i.e., a single stage test) as well as a portion of the test when the test comprises a plurality of different processing steps. For example, the period from activation of the test device to when the device is ready for application of a test fluid can define a stage of the test. Similarly, the period of time from application of a test fluid to completion of processing of the test fluid can define a single stage. Even further test stages can be encompassed, and the present disclosure is not intended to be limited to only the example embodiments discussed.

Known test devices have incorporated liquid crystal display (LCD) screens that allow for limited functionality. Such devices can allow for display of a limited set of symbols—e.g., “YES,” “NO,” a clock face, or a question mark, and these symbols are displayed as static, binary images (i.e., black on a light or white background). Devices according to the present disclosure provide advantages over known devices in that visual message display can be interpreted by a user as being more informative and allowing for better differentiation of test results as well as being more aesthetically pleasing. This can be achieved through one or more of a color digital display, a digital display adapted for simulating motion, and a digital display adapted for progressive illumination (or darkening).

A display according to the present disclosure can be defined according to a variety of embodiments with different levels of complexity. In one example embodiment, a negative LCD display scheme can be utilized. In particular, the LCD screen can comprise a plurality of liquid crystal display characters positioned in an opaque field or background. The background and the LCD characters can be a substantially identical opaque hue. The LCD characters in this embodiment can be switchable between the opaque hue and transparent. When opaque, the characters substantially blend into the background or surrounding opaque field. When transparent, a visible light path through the characters is revealed allowing for viewing of the characters and interpreting their meaning in relation to the assay. Visibility of the light path can arise from external light reflected from a reflective surface of the display underlying the characters or through internally generated light (e.g., from back lighting, such as light emitting diode) transmitted through the transparent character. The characters can be viewed in visible color through use of an appropriate color filter positioned in the display. The same color filter can be used for all of the characters visible in the display, or different color filters can be used for two or more of the characters. If desired, colored LEDs can be used as backlighting to provide the visible color to the transparent characters.

The characters shown on the display of the present device can encompass a variety of messages that can be interpreted by a user of the device. A display character can be a letter, a number, one or more words, a symbol, a picture, or any combination thereof. In particular embodiments, variable text characters covering the entire English alphabet (or further language if desirable) can be provided.

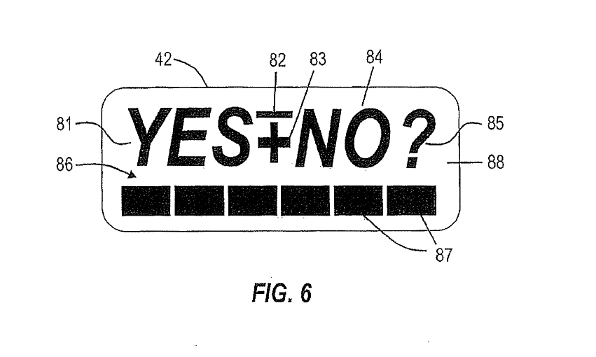

A display according to the present disclosure can be static in nature in that a specific, displayed character is location specific on the display screen and cannot be variably displayed at different locations on the display screen. An example of a static display is shown in FIG. 6, wherein a display screen 42 defines the following characters: a “yes” character 81; a negative character 82; a positive character 83; a “no” character 84; a question character 85; and a status bar 86 defined by a plurality of status segments 87. The characters are positioned within a background 88 that is lightly tinted in grayscale, which is typical of many LCD displays arising from the physical structure of the display. Each character can only be displayed at its single, defined location. Although all characters are shown in FIG. 6, it is understood that the characters are not all simultaneously visible and rather are made visible only when appropriate to display the requisite information regarding the underlying test. The characters are illustrated in black. According to the present disclosure, however, static displays preferably can be provided in a visible color as described herein to improve viewability of the display by a user. For example, the characters can be displayed in a primary color, such as red, yellow, and blue (or variations or combinations thereof). As an example according to the present disclosure, the background of FIG. 6 can be substantially black, and the various characters, when individually visible, can be shown in the same or different colors.

A display according to the present disclosure also can be dynamic in nature. A dynamic display can differ from a static display in that one character or a plurality of characters can be displayed in a sequence that simulates motion on the display screen. In other words, the display screen is adapted to display one or more characters in a manner that is perceived by the human eye as having motion on the display screen. For example, a static display may be adapted to provide a character that does not change in location and that is illustrative of a clock face with one or two hands, and the static display of the clock is indicative that a test is processing. A dynamic display according to the present disclosure, however, may be adapted to provide a character or a group of characters that is illustrative of a clock face with one or two hands wherein one or both of the hands move around the clock face. Such display is only an illustrative embodiment of the various dynamic displays provided by the present disclosure.

The distinction in the exemplary embodiment is significant in that the clock face on the static display has reduced ability to relay temporal characteristics of the test in progress compared to the dynamic display where, for example, the time left in a processing test may be correlated to the movement of the hand around the clock face, if desired. A dynamic display also can, for example, provide for movement of a character or a group of characters in one or more directions on the screen (including scrolling in one or more directions across the screen). Specifically, text may scroll from right to left across a screen such that the text can be read by a user in a typical left-to-right manner. Similarly, text (such as complete words) may scroll up or down a screen, and such scrolling may be continuous or may pause for a pre-determined time while each word is fully visible on the screen. As a further, non-limiting example, a dynamic display can show a rotating hourglass as illustration of passing time while a test progresses.

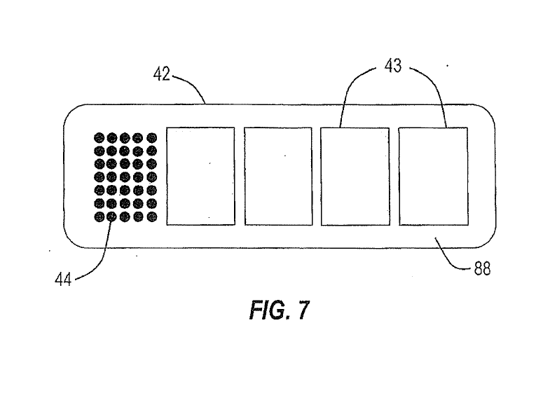

A display useful according to the disclosure particularly can be adapted for rendering variable text characters. As such, the display can comprise a plurality of multi-pixelated fields, each pixelated field being adapted to display a single character at a time (e.g., a single letter or symbol). The pixelated fields can be in a side-by-side arrangement and thus provide for display of a plurality of characters at the same time. Specifically, the number of characters viewable on the display at a single time can be equivalent to the number of multi-pixelated fields present on the display. For example, FIG. 7 illustrates a display screen 42 comprising five multi-pixelated fields 43, each field comprising 35 pixels 44 (or segments) arranged in five vertical columns and seven horizontal rows. In FIG. 7, the fields 43 are illustrated with outlines for ease of references, and no characters are displayed. Further, the left-most field shows details below the screen surface to make visible the individual pixels 44. Each field (as well as each pixel) can be individually addressable and can be adapted to display any character in visible contrast to the background 88 as otherwise described herein. In some embodiments, substantially the entire screen can define a single field formed of a plurality of pixels that can be individually addressable such that the display is adapted to render various characters at any location on the display wherein a sufficient number of pixels are available to define the character.

As seen in FIG. 2, a display screen 42 on a device 10 can be relatively small due to the desirably compact nature of the device. As such, the limited display area can significantly limit the number of characters that can be displayed at one time. This also limits the scope of messages that can be relayed, particularly words and sentences. A dynamic display according to the present disclosure can overcome such limitations by providing for scrolling of characters. For example, in horizontal scrolling, a word or series of words (optionally including other characters) can be made to move across the display by moving each character one position right-to-left. The movement can be at fixed intervals, such as moving one position per second (or greater or lesser interval time to provide for best readability of the displayed message). Accordingly, words and sentences that are otherwise too large to fit in the display area can be made readable according to particular embodiments by moving left-to-right across a reading window. The text thus can provide complete sentences and impart a variety of types of readable information including, for example, instructions, advice, test progress, and other messages. Preferably, the microcontroller or a controller internal to the driver can include an embedded code adapted to direct the display to make viewable the correct characters in the correct sequence, and at the appropriate test stages, so as to form the scrolling text.

In further embodiments of the present disclosure, a display can be adapted to provide progressive illumination of a plurality of characters. In progressive illumination, a plurality of characters can be sequentially arranged and be illuminated or otherwise made viewable on the display screen at pre-determined times. The progression thus can relay temporal information to a user in relation to the status of a test. For example, the progression can count up or count down a time until a test device is prepared for use, a time until a test stage is completed, a time until a test as a whole is completed, and like embodiments. Time periods encompassed by the progressive illumination can be pre-determined or can vary between devices (e.g., depending upon use conditions, volume of sample applied to the device, etc.). A test device according to the disclosure therefore can be defined as being adapted to implement a progressive display count. As noted, the count can be carried out as a count up or as a count down. In particular, the microcontroller of the device can be defined as being adapted to implement the progressive display count.

Progressive illumination according to the present disclosure can encompass one or both of positive progressive illumination and negative progressive illumination. In positive progressive illumination, all characters in the sequence are initially darkened, and the characters are sequentially illuminated until all characters in the sequence are simultaneously illuminated. This may also be referred to as a progressive build. In negative progressive illumination, all characters in the sequence are initially illuminated, and the characters are sequentially darkened until all characters in the sequence are darkened. This also may be referred to as a progressive hide. In some embodiments, such as where motion of a character is perceived, progressive illumination can be an example of dynamic display. The use of the word “illumination” should not be viewed as necessarily limiting the manner in which characters are sequentially made visible. Illumination may arise from reflective lighting of the appropriate portions of the screen to make the appropriate characters visible or may arise from backlighting. Likewise, activation of one or more pixels on a pixelated screen may account for illumination of the desired characters in the sequence. In such manners, the display of the device is thus adapted to display a sequential arrangement of a plurality of characters that can be illuminated (or darkened) in sequence to illustrate progress of a test or test stage and thus provide an approximate time until completion of the test or test stage.

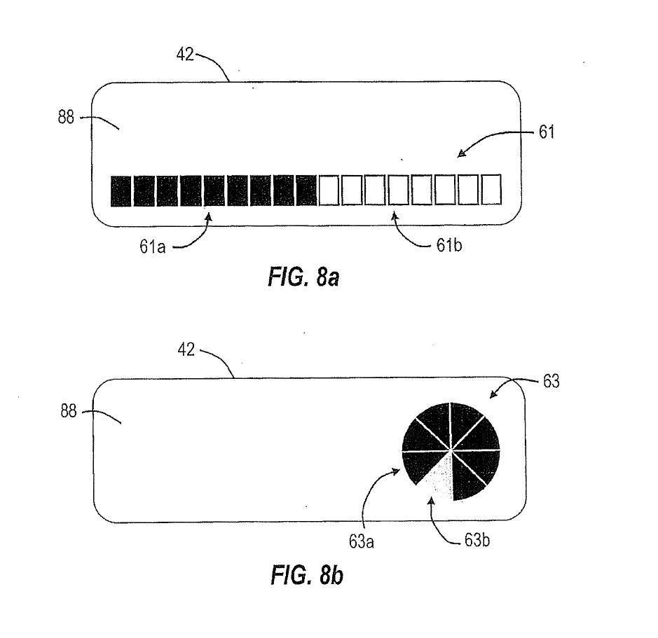

An exemplary embodiment of progressive illumination is shown in FIG. 8a, wherein a display screen 42 is provided with a progress bar positioned near the bottom of the display screen and defined by a sequence of bar sections 61. As illustrated, the progress bar has multiple illuminated bar sections 61a on the left end thereof (shown in black) and multiple darkened (or non-visible) bar sections 61b on the right end thereof (where the outline is shown for illustrative purposes and may not necessarily be visible on the display screen when darkened). The bar sections 61a are considered illuminated in that they are displayed in visible contrast to the background 88 of the display screen 42, which is illustrated in lightly tinted grayscale. The bar sections 61b are considered darkened in that they are not substantially visible in the background 88 of the display screen 42. This can illustrate, for example, the progression of a test wherein the bar sections 61 are initially darkened and are illuminated in sequence from left to right as the test progresses. In use, the background 88 can take on a color, if desired, and the characters, when illuminated, are visible in a color that is clearly distinguishable from the background. In FIG. 8a, the number of illuminated bar sections 61a can be interpreted as indicating that more than half of the time needed for the test to reach completion has passed. A greater or lesser number of progress bars may be used, and the size and shape of the progress bars also can be varied as desired. For example, the bar sections can increase in size moving from right to left to further emphasize progression of the test. The embodiments illustrated in relation to FIG. 8a likewise can be adapted for progressive hide (or darkening) wherein all of the bar sections 61 are illuminated when the test begins, and the bar sections darken in sequence as the test progresses so as to provide a countdown of time remaining in the test. Similarly, all of the bar sections 61 may flash (i.e., alternate rapidly between illuminated and darkened) when the test begins, and the bar sections change in sequence from flashing to constant illumination (or from flashing to constantly darkened) to indicate progression of the test. In alternative embodiments, only one bar section 61 may be illuminated at a given time, and the bar section that is illuminated can progress in sequence (e.g., from left to right) as the test progresses. Likewise, a single bar section 61 may scroll across the display screen 42.

In FIG. 8a, the upper portion of the display screen 42 can be reserved for display of further characters that can be viewable at different stages of the test. In particular, the upper portion of the display screen 42 may be adapted for scrolling text as otherwise described herein. As such, a display according to the present disclosure can also be bifurcated or otherwise adapted to provide for different types of displays in different areas or sections of the display. Likewise, the position of the progress on the display screen can vary.

Another exemplary embodiment of progressive illumination is shown in FIG. 8b wherein a display screen 42 is provided with a progress wheel defined by a sequence of wedges 63. As illustrated, the progress wheel has illuminated wheel sections 63a and darkened wheel sections 63b. The wheel sections 63a are considered illuminated in that they are displayed in visible contrast to the background 88 of the display screen 42, which is illustrated in lightly tinted grayscale. The wheel sections 63b are considered darkened in that they are not substantially visible in the background 88 of the display screen 42. This can illustrate, for example, the progression of a test wherein the wheel sections 63 are initially darkened and are illuminated in sequence around the wheel (either clockwise or counter clockwise) as the test progresses. In FIG. 8b, the number of illuminated wheel sections 63a can be interpreted as indicating that the test or a stage thereof is close to completion. A greater or lesser number of wheel sections may be used, and the size and shape of the wheel sections also can be varied as desired. The embodiments illustrated in relation to FIG. 8b likewise can be adapted for progressive hide wherein all of the wheel sections 63 are illuminated when the test begins, and the wheel sections darken in sequence as the test progresses so as to provide a countdown of time remaining in the test. Similarly, all of the wheel sections 63 may flash (i.e., alternate rapidly between illuminated and darkened) when the test begins, and the wheel sections change in sequence from flashing to constant illumination (or from flashing to constantly darkened) to indicate progression of the test. In alternative embodiments, only one wheel section 63 may be illuminated at a given time, and the wheel section that is illuminated can progress in sequence (e.g., clockwise or counter clockwise) as the test progresses. Likewise, a single wheel section 63 may cycle around the wheel on the display screen 42.

The embodiments of progressive illumination described in relation to FIG. 8a and FIG. 8b are intended to be exemplary embodiments. Thus, such embodiments should not be viewed as limiting and, in fact, variations of progression schemes illustrated are encompassed by the present disclosure. For example: a progress bar may be arranged in a top to bottom manner rather than a side to side manner; individual segments of a progress indicator may graduate in size; a progress indicator may be a sequence of individual segments that form an outline of a circle, oval, or other shape; and segments of a progress indicator may be in a color rather than black and may have different colors for different segments.

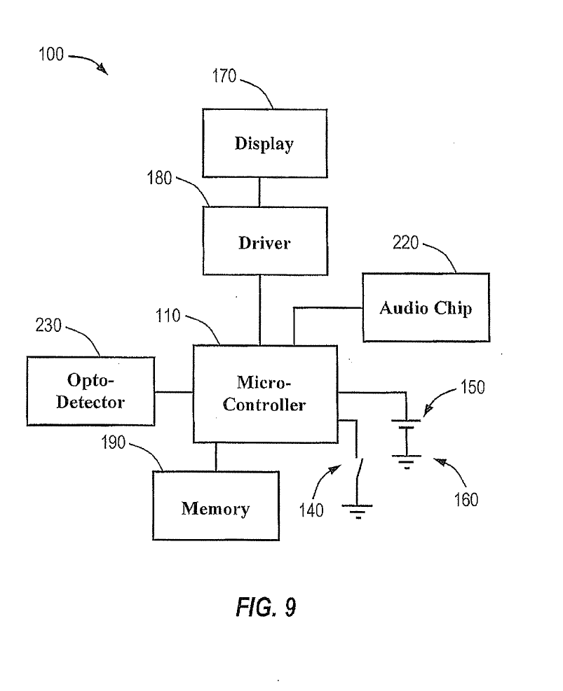

Another example embodiment of a schematic of operational components of an electronic communication circuit 100 useful according to the present disclosure is shown in FIG. 9. As seen therein, the communication circuit 100 includes components that provide for displays with increased complexity and richness of output. The communication circuit again includes a microcontroller 110 that can execute the functions otherwise described herein, but the circuit optionally can include a driver 180 that can comprise a dedicated internal circuit, which can be a separate microcontroller. A segment display driver chip (or text display driver chip) useful according to the disclosure particularly can be one capable of converting input serial or parallel data containing character codes into drive signals for individual display segments on the display 170. The driver 180, when present, can be a separate component or can be combined with the display 170. The driver can have integrated RAM or ROM including programming for a variety of characters to be displayed by the test device. Alternatively, the RAM or ROM of the microcontroller 110 can include the necessary programming to direct the appropriate visual output. Still further, an additional memory component 190 can be included for storage of, for example, necessary coding for the variety of characters and combinations thereof to be displayed by the disclosed devices and correspond to one or more indicia of operation of the test member. Such coding also can be present (in part or in whole) in a memory component internal to the microcontroller 110 or the driver 180. A non-limiting example of such coding is coding in ASCII format for one or more words, phrases, and/or sentences in one or more languages.

Memory size for an electronic communication circuit useful according to the present disclosure can vary based upon the complexity of display desired, including the number of characters to be displayed, the nature of the display (static versus dynamic), individual character size (i.e., number of pixels), and resolution. For example, ten uncompressed images at 8 bits per color on a display of 120×160 pixels can require up to about 1-2 MB of memory. Compressing the images can reduce memory requirements, such as by a factor of ten. The microcontroller 110 can provide the output signals to the driver 180 to direct the appearing of the one or more visual characters to be output by the display 170.

The display 170 can utilize a variety of technology platforms that are adapted for providing enhanced viewing characteristics as described herein. For example, as discussed above, the display can be an LCD. Preferably, LCD technology used in the display is adapted for viewing of one or more characters in color. In some embodiments, a display 170 useful according to the present disclosure can comprise a plurality of addressable segments (i.e., pixels) that can be individually controlled, including being color-variable and/or being present as a plurality of multi-pixelated fields. In such embodiments, the microcontroller 110 can be adapted for individual control of a plurality of pixels. For example, a flash microcontroller such as PIC16F1826 (available from Microchip Technology, Inc.) can provide technical specifications useful in variable control of a plurality of individual pixels on a display.

In certain embodiments, a display useful according to the present disclosure can comprise nematic LCD, super-twist nematic (STN) LCD, thin film transistor (TFT), light emitting diode (LED), or organic light emitting diode (OLED) components. Combinations of such components (e.g., combination of an LCD component with a TFT component) also are encompassed. As will be recognized by the skilled artisan, such components can be particularly beneficial in providing for a plurality of individually addressable pixels that can be powered over an intensity range from completely off to fully attainable brightness and can be viewed over a range of different colors. Such display technology can allow for a variety of combinations of displayed characters and/or display colors in either static or dynamic fashion or via progressive illumination.

As illustrated in FIG. 9, an electronic communication circuit 100 can provide for a wide array of viewable outputs from the display 170. In particular, at defined intervals or test stages, the microcontroller 110 can send pre-determined character codes (e.g., for display of pre-determined symbols, letters, numbers, words, or sentences) in a pre-determined sequence to the display 170 and/or driver 180 to cause a variety of messages to be displayed for viewing by a user. For example, the assay initiation routine (e.g., triggered by removal of the cap 14) can signal the microcontroller to provide an output to the display forming a viewable character or combination of characters, words and combinations of words, that provides one or more instructions to the user in relation to steps necessary to carry out the assay with the test device. Letters, numbers, symbols, or pictures relaying other types of information to the user also can be provided. Once the assay has been initiated by the user (i.e., through application of fluid sample), the microcontroller can instruct the display to provide viewable indication that the test has been started. In particular, the communication circuit 100 can include an opto-detector 230 comprised of a light source, such as one or more LEDs and one or more light sensors that can be calibrated to detect indicia of operation of the assay at the sample receiving member 12, the release medium 58, or the capture medium 54. Alternatively (or in addition), the communication circuit also can include a moisture sensor. Viewably displayed indication of the assay start can be as described above. The electronic communication circuit also can be adapted to output viewable information in relation to the test time elapsed and/or remaining for the assay to reach completion (generally less than about 5 minutes from the time of fluid sample application). As non-limiting examples, a digital timer can be displayed counting up or down, a sequence of shapes or symbols can be added or removed as indication of passing time (e.g., progressive illumination), or a clock face can be displayed with a moving hand (e.g., dynamic display). Alternatively, words and sentences can be displayed during the processing time to provide reassurance to the user of proper function. Moreover, words and messages can include further instructions related to the assay, marketing messages, or the like. When the assay has reached completion, the display can receive and execute instructions for output indicating that the test time has elapsed and can also provide visual indication of the assay results, such as a symbol or words or sentences relaying a message recognizable as either a positive or negative test result. If desired, the communication circuit can include a light switch 140 adapted to enable or disable backlighting on the display 170 to further improve viewability of the displayed characters.

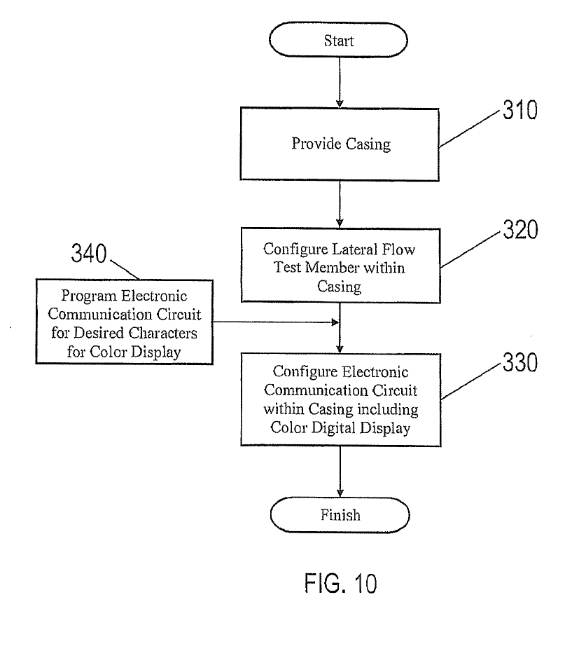

The present disclosure also provides for methods for providing one or more indicia of operation of a lateral flow assay to a user. In particular, the methods can include steps for the manufacture of a test device, such as otherwise described herein. An exemplary embodiment of the methods of the disclosure is shown in FIG. 10. For example, a test device casing can be provided in step 310. The components of a lateral flow assay can be incorporated into the casing in step 320. Moreover, elements forming an electronic communication circuit also can be incorporated into the casing in step 330. Specifically, a microcontroller, one or more components adapted to provide input signals to the microcontroller relating to the test member, and a color digital display can be configured in the casing. In specific embodiments, the methods can comprise programming the microcontroller with coding that defines a response to a defined input signal upon which the microcontroller responds by signaling the color digital display to make viewable a defined character or combination of characters. This programming is illustrated in step 340. Such step can be carried out through application of the appropriate hardware and/or software that is adapted to create stored commands and execution routines within the communication circuit. The method further can comprise selection of appropriate characters to correspond to the desired indicia of operation of the diagnostic test device.

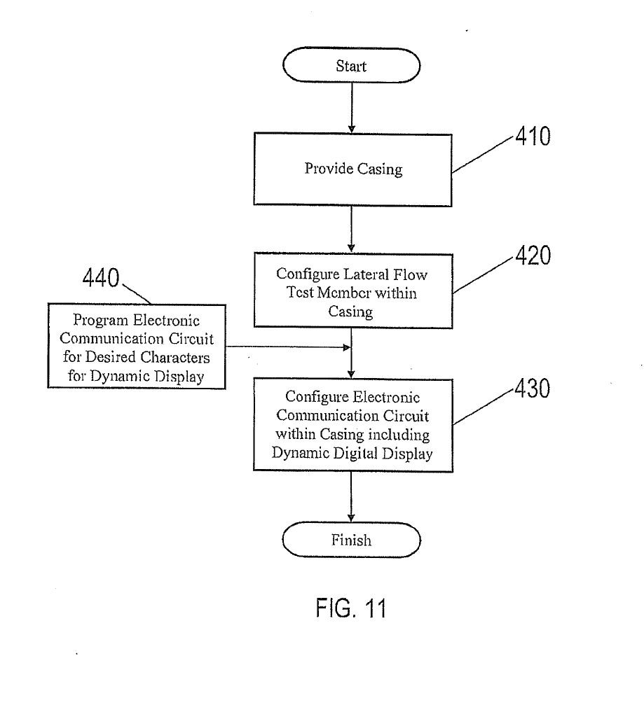

Another exemplary embodiment of the methods of the disclosure is shown in FIG. 11. Specifically, a test device casing can be provided in step 410. The components of a lateral flow assay can be incorporated into the casing in step 420. Moreover, elements forming an electronic communication circuit also can be incorporated into the casing in step 430. Specifically, a microcontroller, one or more components adapted to provide input signals to the microcontroller relating to the test member, and a dynamic digital display can be configured in the casing. In specific embodiments, the methods can comprise programming the microcontroller with coding that defines a response to a defined input signal upon which the microcontroller responds by signaling the dynamic digital display to make viewable a defined character or combination of characters, including providing for scrolling text. This programming is illustrated in step 440. Such step can be carried out through application of the appropriate hardware and/or software that is adapted to create stored commands and execution routines within the communication circuit. The method further can comprise selection of appropriate words or characters to correspond to the desired indicia of operation of the diagnostic test device.

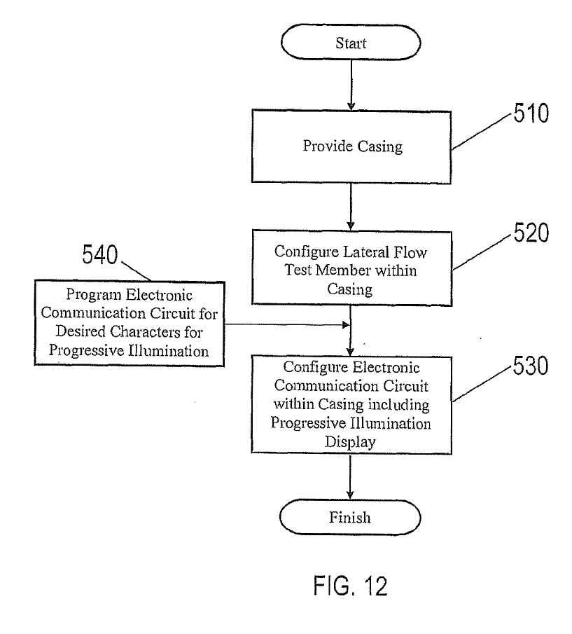

A further exemplary embodiment of the methods of the disclosure is shown in FIG. 12. Specifically, a test device casing can be provided in step 510. The components of a lateral flow assay can be incorporated into the casing in step 520. Moreover, elements forming an electronic communication circuit also can be incorporated into the casing in step 530. Specifically, a digital display adapted for progressive illumination of a plurality of characters, a microcontroller adapted to signal the digital display to progressively illuminate or darken the characters in a defined sequence, and one or more components adapted to provide input signals to the microcontroller relating to the test member can be configured in the casing. In specific embodiments, the methods can comprise programming the microcontroller with coding that defines a response to a defined input signal upon which the microcontroller responds by signaling the digital display to progressively illuminate or darken a sequence of characters. This programming is illustrated in step 540. Such step can be carried out through application of the appropriate hardware and/or software that is adapted to create stored commands and execution routines within the communication circuit. The method further can comprise selection of desired characters, sequence or illumination or darkening, and time interval defining the progression.

Many modifications and other embodiments of the disclosure set forth herein will come to mind to one skilled in the art to which these disclosure pertain having the benefit of the teachings presented in the foregoing descriptions. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A diagnostic test device comprising:
a test member; and
an electronic communication circuit adapted to provide one or more indicia of operation of the test member to a user;
wherein the electronic communication circuit includes a color digital display.

2. The diagnostic test device of claim 1, wherein the color digital display comprises a plurality of color-variable pixels.

3. The diagnostic test device of claim 1, wherein the color digital display comprises one or more light emitting diode (LED).

4-5. (canceled)

6. The diagnostic test device of claim 1, wherein the color digital display comprises a liquid crystal display (LCD).

7-10. (canceled)

11. The diagnostic test device of claim 6, wherein the LCD comprises backlighting.

12. The diagnostic test device of claim 1, wherein the electronic communication circuit comprises a microcontroller.

13-23. (canceled)

24. The diagnostic test device of claim 1, wherein the electronic communication circuit further includes an audio output component.

25. (canceled)

26. The diagnostic test device of claim 1, wherein the test device is adapted to detect the presence of an analyte in a fluid sample applied to the test member.

27-28. (canceled)

29. A diagnostic test device comprising:
a test member; and
an electronic communication circuit adapted to provide one or more indicia of operation of the test member to a user;
wherein the electronic communication circuit includes a dynamic digital display.

30. The diagnostic test device of claim 29, wherein the dynamic digital is adapted to render variable text characters.

31. The diagnostic test device of claim 29, wherein the dynamic digital display is adapted to display one or more characters in a sequence that simulates motion on the display.

32. (canceled)

33. The diagnostic test device of claim 31, wherein the one or more characters define a word or a string of words.

34. (canceled)

35. The diagnostic test device of claim 31, wherein the sequence in which the characters are displayed defines scrolling of the characters on the display.

36-51. (canceled)

52. A diagnostic test device comprising:
a test member; and
an electronic communication circuit adapted to provide one or more indicia of operation of the test member to a user;
wherein the electronic communication circuit comprises: a digital display adapted for progressive illumination of a plurality of characters; and a microcontroller adapted to signal the digital display to progressively illuminate or darken the characters in a defined sequence.

53-57. (canceled)

58. The diagnostic test device of claim 52, wherein the digital display is adapted to display the plurality of characters in color.

59-72. (canceled)

73. A method for providing one or more indicia of operation of a test device to a user, the method comprising combining, in a single casing:
a test member; and
an electronic communication circuit that includes:
a microcontroller;
one or more components adapted to provide input signals to the microcontroller relating to the test member; and
a color digital display.

74. The method of claim 73, further, comprising programming the microcontroller to respond to a defined input signal by signaling the color digital display to make visible one or more color characters defining the one or more indicia of operation of the test.

75. The method of claim 74, wherein the character is selected from the group consisting of letters, numbers, symbols, pictures, and combinations thereof.

76. A method for providing one or more indicia of operation of a test device to a user, the method comprising combining, in a single casing:
a test member; and
an electronic communication circuit that includes:
a microcontroller;
one or more components adapted to provide input signals to the microcontroller relating to the test member; and
a dynamic digital display.

77. The method of claim 76, wherein the dynamic digital display comprises a plurality of multi-pixelated fields.

78-79. (canceled)

80. A method for providing one or more indicia of operation of a test device to a user, the method comprising combining, in a single casing:
a test member; and
an electronic communication circuit that includes:
a digital display adapted for progressive illumination of a plurality of characters;
a microcontroller adapted to signal the digital display to progressively illuminate or darken the characters in a defined sequence; and
one or more components adapted to provide input signals to the microcontroller relating to the test member.

81-82. (canceled)

83. The method of claim 80, wherein the input signal defines the start of a test or a stage thereof, and wherein the method further comprises programming the microcontroller to respond to the input signal by signaling the digital display to progressively illuminate or darken the characters over a time period defined by the amount of time for completion of the test or the stage thereof.
